(19) United States
(12) Patent Application Publication
(10) Pub. No.: US 2022/0418157 A1
(43) Pub. Date: Dec. 29, 2022

(54) APPARATUS AND METHOD FOR SURFACE DISINFECTION USING UV LIGHT

(71) Applicants: Jason DuBose, Corvallis, OR (US); Megan Pham, Wilsonville, OR (US)

(72) Inventors: Jason DuBose, Corvallis, OR (US); Megan Pham, Wilsonville, OR (US)

(21) Appl. No.: 17/853,513

(22) Filed: Jun. 29, 2022

Related U.S. Application Data
(60) Provisional application No. 63/216,089, filed on Jun. 29, 2021.

(51) Int. Cl. H05K 7/20; H01L 25/075; H01L 33/64; A61L 2/26; A61L 2/10

(52) U.S. Cl. CPC H05K 7/20263; H01L 25/0753; H01L 33/64; A61L 2/26; A61L 2/10; A61L 2202/11

(57) ABSTRACT

A device for disinfecting surfaces is provided. One embodiment has a plurality of UV light (energy) emitting LEDs residing in an enclosure, wherein emitted UV energy passes through a lens onto a surface being disinfected; a heat dissipator that receives heat generated by the UV emitting LEDs; and a fluid moving device. The enclosure has a fluid heating passageway that is in fluid contact with the heat dissipator, has a transfer passageway with a distal end that is fluidly coupled to a proximal end of the fluid heating passageway, and has a return passageway that is fluidly coupled to a proximal end of the transfer passageway and that is fluidly coupled to a distal end of the fluid heating passageway. During operation, the fluid moving device operates to circulate a cooling fluid through the fluid heating passageway, the transfer passageway, and the return passageway. Heat transfers to the ambient environment.

### PRIORITY CLAIM

This application claims priority to copending U.S. Provisional Application, Ser. No. 63/216,089, filed on Jun. 29, 2021, entitled Apparatus and Method For Surface Illumination, which is hereby incorporated by reference in its entirety for all purposes.

### BACKGROUND OF THE INVENTION

Various devices are known to disinfect, decontaminate, cure and/or illuminate a surface. More particularly, high power ultraviolet (UV) spectrum light emitting diode (LED) devices are known for use in disinfecting, decontaminating, curing and/or illuminating surfaces. However, the size and/or configuration of such LED devices are not particularly well suited to many potential applications.

For example, but not limited to, UV spectrum LED devices may be used to disinfect, decontaminate, cure and/or illuminate plants. However, plants are relatively fragile and may be prone to damage if a large, cumbersome legacy UV spectrum LED device is used on plants. Further, because of the density of some plant foliage, it is nearly impossible to disinfect, decontaminate, cure and/or illuminate all plant surfaces, particularly those surfaces of a plant that are well inside the interior region of the plant's foliage.

Accordingly, in the arts of UV spectrum LED devices, there is a need in the arts for improved methods, apparatus, and systems for a UV spectrum LED device that is particularly suitable for use with plants.

### SUMMARY OF THE INVENTION

Embodiments of a device for disinfecting surfaces is provided. One embodiment has a plurality of UV emitting LEDs residing in an enclosure, wherein emitted UV light (energy) passes through a lens onto a surface that is being disinfected; a heat dissipator receives heat generated by the UV emitting LEDs; and a fluid moving device. The enclosure has a fluid heating passageway that is in fluid contact with the heat dissipator and has a transfer passageway with a distal end that is fluidly coupled to a proximal end of the fluid heating passageway, and has a return passageway that is fluidly coupled to a proximal end of the transfer passageway and that is fluidly coupled to a distal end of the fluid heating passageway. During operation of the device to emit UV energy, the fluid moving device operates to circulate a cooling fluid through the fluid heating passageway, the transfer passageway, and the return passageway. Heat is transferred from the cooling fluid to the ambient environment.

### BRIEF DESCRIPTION OF THE DRAWINGS

The components in the drawings are not necessarily to scale relative to each other. Like reference numerals designate corresponding parts throughout the several views.

FIG. 1 is a perspective view of a UV spectrum LED device.

FIG. 2 is a cross sectional view of the UV spectrum LED device.

FIG. 3 is a longitudinal cross-sectional view of the UV spectrum LED device.

FIG. 4 is a perspective view of a UV spectrum LED device employing four of arrays of UV emitting LEDs.

FIG. 5 is a perspective view of a UV spectrum LED device employing two of arrays of UV emitting LEDs and that is securable to a surface of an object.

FIG. 6 is a perspective view of a UV spectrum LED device employing one array of UV emitting LEDs and that is securable to a surface of an object.

FIG. 7 is a longitudinal cross-sectional view of the UV spectrum LED device of FIG. 6.

FIG. 8 is a perspective view of a UV spectrum LED device fluidly coupled to a cooling device.

### DETAILED DESCRIPTION

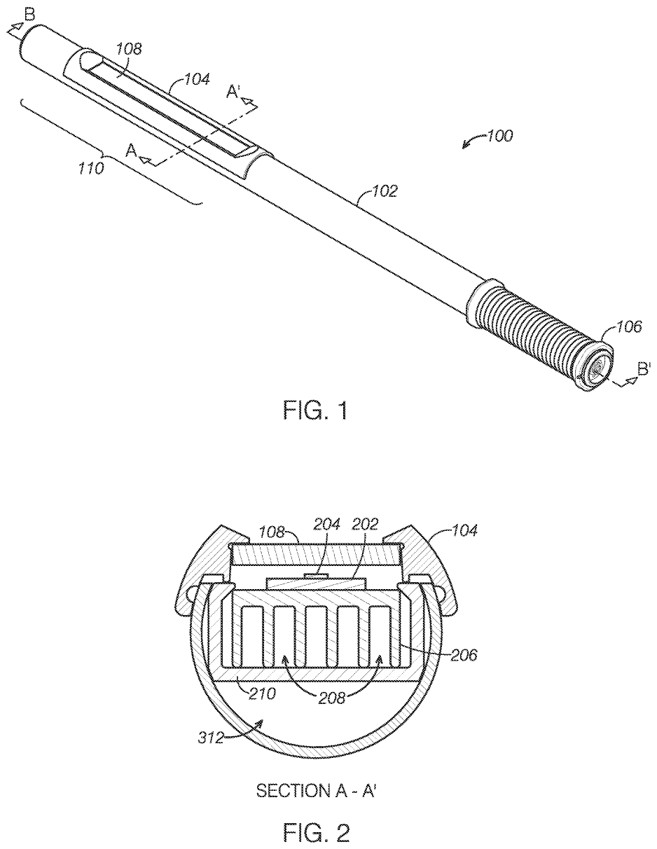

FIG. 1 is a perspective view of an ultraviolet (UV) spectrum light emitting diode (LED) device 100. The non-limiting example UV spectrum LED device 100 is a slim, elongated LED device that emits UV spectrum light energy using a plurality of light emitting diodes in a hermetically sealed, hygienic package that can be easily cleaned, disinfected, and/or sanitized. Other embodiments may emit different wavelengths of light energy for disinfecting, decontaminating, curing and/or illuminating surfaces. Any surface may be disinfected, decontaminated, cured and/or illuminated.

However, embodiments of the UV spectrum LED device 100 are particularly well suited for disinfecting, decontaminating, curing and/or illuminating surfaces of plants and/or other hard to reach surfaces. The long and slender configuration permits the UV spectrum LED device 100 to be inserted into the foliage of a plant in a very non-invasive manner, with little or no damage to the plant's foliage, which permits illumination of such hard-to-reach places within the plant.

Further, UV spectrum LED devices generate significant amounts of heat when in use. Such heat can be damaging to plant tissue. Embodiments of the UV spectrum LED device 100 employ a novel apparatus and method to draw out the generated heat away from the portion of the UV spectrum LED device 100 that is inserted into the plant's foliage, thereby reducing or eliminating potential heat damage to the plant.

Heat is known to degrade LEDs 204. Accordingly, an unexpected benefit of cooling the LEDs 204 using a fluid moving through the heating passageway 208 is to increase the operational life of the UV emitting LEDs 204.

A preferred example embodiment provides a hermetically sealed and/or waterproof UV spectrum LED device 100. An advantage of a hermetically sealed embodiment is that after use, the exterior of the UV spectrum LED device 100 can be cleaned, disinfected and/or sanitized using water and/or a cleaning agent to prevent cross contamination between subsequent uses. The hermetically sealed embodiment may even be dipped into a container holding a disinfectant fluid. For example, if the UV spectrum LED device 100 is used to kill undesirable mold on plants, the UV spectrum LED device 100 can be disinfected between each use.

The disclosed systems and methods for disinfecting, decontaminating, curing and/or illuminating surfaces using the novel UV spectrum LED device 100 will become better understood through review of the following detailed description in conjunction with the figures. The detailed description and figures provide examples of the various inventions described herein. Those skilled in the art will understand that the disclosed examples may be varied, modified, and altered without departing from the scope of the inventions described herein. Many variations are contemplated for different applications and design considerations, however, for the sake of brevity, each and every contemplated variation is not individually described in the following detailed description.

Throughout the following detailed description, a variety of examples for systems and methods for using the UV spectrum LED device 100 are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder but may have one or more deviations from a true cylinder.

“Comprising,” “including,” and “having” (and conjugations thereof) are used interchangeably to mean including but not necessarily limited to, and are open-ended terms not intended to exclude additional, elements or method steps not expressly recited.

Terms such as “first”, “second”, and “third” are used to distinguish or identify various members of a group, or the like, and are not intended to denote a serial, chronological, or numerical limitation.

“Coupled” means connected, either permanently or releasably, whether directly or indirectly through intervening components. “Secured to” means directly connected without intervening components.

“Communicatively coupled” means that an electronic device exchanges information with another electronic device, either wirelessly or with a wire-based connector, whether directly or indirectly through a communication network. “Controllably coupled” means that an electronic device controls operation of another electronic device.

Returning to FIG. 1, the example UV spectrum LED device 100 comprises an enclosure 102, a lens housing 104 (proximate to a distal end of the UV spectrum LED device), and a grip or securing mechanism 106 (proximate to a proximate end of the UV spectrum LED device 100). The lens housing 104 secures a lens 108, wherein a plurality of interior LEDs emits the UV spectrum energy through the lens 108.

Any suitable length of the UV spectrum LED device 100 may be used in the various embodiments, but preferably, a length that permits the user to penetrate into difficult to reach places within the interior of a plant's foliage. If the UV spectrum LED device 100 is used to disinfect, decontaminate, cure and/or illuminate other surfaces, the length may be defied to reach such difficult-to-reach surfaces. In some embodiments, the length of the enclosure 102 may be adjustable, such as by using expandable/collapsible telescoping tubing for a portion of the enclosure 102. For example, a telescoping enclosure 102 embodiment may be particularly desirable for disinfecting, decontaminating, curing and/or illuminating surfaces of trees.

The size of the lens 108 may be defined based on the particular surface that is to be disinfected, decontaminated, cured and/or illuminated. For example, a relatively long length of the lens 108 (and the underlying LEDs) may be used to access foliage of relatively large plants. In contrast, a shorter length of the lens may be used when plants are relatively small.

Depending upon the particular task of the disinfecting, decontaminating, curing and/or illuminating, the surface area of the lens 108 may be defined so that the underlying LEDs emit a desired amount and/or intensity of radiation. The effectiveness of the UV spectrum LED device 100 will depend upon the nature of the surface, the type of organisms that are to be killed, the illumination intensity of the emitted UV energy, and the duration of time that the UV spectrum LED device 100 is illuminating the surface that is being disinfected, decontaminated, cured and/or illuminated.

In some applications, a relatively large amount of UV radiation or other spectrum radiation is needed to disinfect, decontaminate, cure and/or illuminate a particular surface. Accordingly, the lens 108 may be relatively wide and long. Further, the lens 108 may be configured to direct and/or focus the emitted radiation from the underlying LEDs. Some embodiments may include a reflective surface underneath or in proximity to the bottom of the UV spectrum LED device 100 to reflect any incident UV radiation outward through the lens 108.

Any suitable grip mechanism 106 may be used in the various embodiments. When the mechanism 106 is configured to be grasped by a user, the outside of the mechanism 106 may be covered with a comfortable, heat insulative, and/or water-resistant material. The mechanism 106 length may be defined for grasping by one of the user's hands, or for grasping by both of the user's hands. Alternatively, the mechanism 106 may facilitate mounting of an embodiment of the UV spectrum LED device 100 to a surface or other object.

In a preferred embodiment, the UV spectrum LED device 100 is cylindrical. However, any suitable cross-sectional shape may be used, including a square, rectangle, oval, or the like. An example embodiment has a length-to-cross-sectional area ratio greater than 17:1, and a thermal dissipation greater than 1.08 watts per inch of length of the conductive portion of the enclosure 102. Any suitable dimensions may be used in the various embodiments. Some embodiments may employ a fin or other heat dissipating structure, or device on the outside surface of the enclosure 102 to facilitate heat dissipation from the UV spectrum LED device 100.

FIG. 2 is a cross sectional view of the UV spectrum LED device 100 at the lens housing 104 (along the A-A′ plane). A light-emitting module 202 secures a plurality of UV emitting LEDs 204 below the lens 108. In a preferred embodiment, the LEDs 204 emit ultraviolet (UV) spectrum energy. In alternative embodiments, the LEDs 204 may emit energy in another spectrum(s). To achieve a desired amount of emitted UV light energy, a plurality of individual UV emitting LEDs 204 may be arranged as an array on a substrate, such as the light-emitting module 202.

Optionally, one or more of the UV emitting LEDs 204 may be configured to concurrently emit energy of a different spectrum. For example, but not limited to, one or more of the LEDs 204 may be configured to emit white light for illumination purposes while the UV energy is being emitted for sterilization of a surface.

Below the light-emitting module 202 is a heat dissipator 206 (heat sink) that is in thermal communication with the light-emitting module 202. Heat generated by the LEDs 204 is transferred from the light-emitting module 202 to the heat dissipator 206. A heating passageway 208 permits flow of a fluid to transfer the heat collected by the heat dissipator 206 away from the light-emitting module 202. The fluid may be a gas, such as, but not limited to air, or may be a liquid coolant.

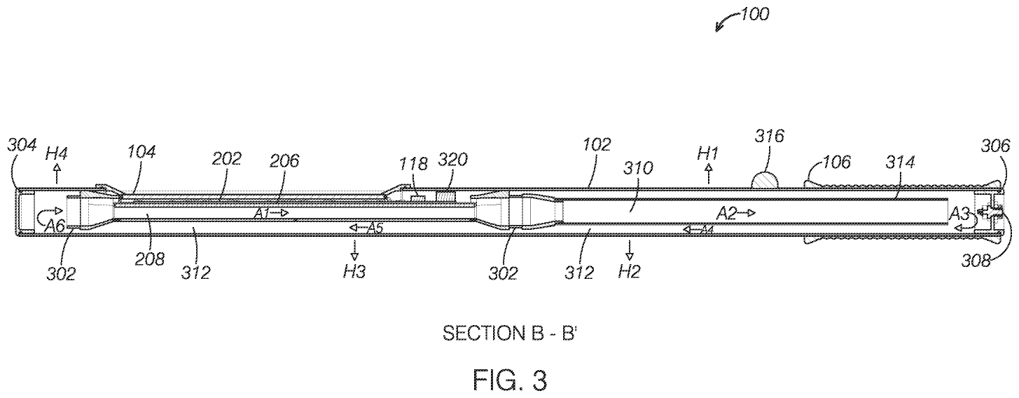

FIG. 3 is a longitudinal cross-sectional view of the UV spectrum LED device 100. At least one fluid moving device 302 is used to move a cooling fluid through the fluid heating passageway 208 so that heat drawn from the light-emitting module 202 by the heat dissipator 206 can be transferred away from the light-emitting module 202 and the distal end portion 110 of the enclosure 102. The example embodiment employs air as the cooling fluid. The cooling fluid circulates along the path denoted with directional arrows (“Ai→”) in FIG. 3. As noted herein, the cooling fluid may be a suitable coolant liquid.

In an example embodiment, an end cap 304 fluidly seals the distal end of the hollow tubular enclosure 102, thus sealing the proximal end of the UV spectrum LED device 100. Additionally, a connector end cap 306 fluidly seals the proximal end of the hollow tubular enclosure 102. The connector end cap 306 provides electrical power connectors 308 so that the UV spectrum LED device 100 can receive power for operation. In a preferred embodiment, the connector end cap 306 can be electrically coupled to a power source by an electrical power cord or the like. Alternatively, or additionally, a battery supply residing in the interior of the enclosure 102 and/or coupled to the UV spectrum LED device 100 may provide power

In practice, the user turns the UV spectrum LED device 100 on so that UV light is emitted by the UV emitting LEDs 204. Heat generated by the UV emitting LEDs 204 is transferred to the heat dissipator 206. The cooling fluid residing in the fluid heating passageway 208 is then heated. The heated fluid is then moved out of the heating passageway 208 (see A1) and into a transfer passageway 310 (see A2). As the heated fluid is transported through the transfer passageway 310, heat from the heated fluid may then be conductively transferred out to the ambient environment (see H1).

The heated fluid, which is now beginning to cool, is then transferred from the transfer passageway 310 to the return passageway 312 (see A3). As the fluid is transported through the return passageway 312 (see A4 and A5) further cooling of the cooling fluid occurs (see H2 and H3) as heat is transferred to the ambient environment. After traversing the length of the return passageway 312, the cooled fluid returns into the heating passageway 208 (see A6). The distal end of the enclosure 102 also allows conductive heat transfer (see H4). Other embodiments may be thermally insulated so that the distal end of the enclosure 102 does not get hot. In some embodiments, the UV emitting distal end portion 110 may be thermally insulated along the corresponding portion of the return passageway 312 (so that heat dissipation at H3 does not occur).

A fluid barrier 210 (FIG. 2) fluidly separates the heating passageway 208 and the return passageway 312 so that the cooling fluid in the heating passageway 208 can be transported over the heat dissipator 206. A second fluid barrier 314 also fluidly separates the return passageway 312 and the transfer passageway 310. Accordingly, the cooling fluid can circulate through the passageways 208, 310, 312 without mixing. In an example embodiment, the barrier structure may be a tube that is inserted into the interior of the enclosure 102. Here, the distal end of the tube is fluidly coupled and sealed with the exit of the heating passageway 208.

An unexpected advantage of using a circulating flow of the cooling fluid to dissipate heat from the light-emitting module 202 is that heat sensitive surfaces, such as plant leaves or fruit, will not become heat damaged during a disinfecting, decontaminating, curing and/or illuminating process. In an example embodiment, the circulating flow of the cooling fluid conductively transfers the heat to the exterior surface of the enclosure 102, where the heat is dissipated into the ambient environment in a radiant or convective manner.

In the non-limiting example embodiment, two or more fluid moving devices 302 are employed at the illustrated locations. If the working coolant fluid is air or another gas, the fluid moving devices 302 may be fans. If the working coolant fluid is a liquid, the fluid moving devices 302 may be pumps. Alternative embodiments may use any suitable number of fluid moving devices 302 at desired locations along the circulating fluid flow path defined by the heating passageway 208, the transfer passageway 310, and the return passageway 312 that cooperatively transport and retain the cooling fluid medium.

In an alternative embodiment, the end cap 304 and a fluid moving device 302 may be integrated together or may be located in proximity to each other. Alternatively, or additionally, the connector end cap 306 and a fluid moving device 302 may be integrated together or may be located in proximity to each other. In operation, cool ambient air is drawn into the passageway 208 via the end cap 304. The proximal end of the connector end cap 306 permits exit of the warmed air from the UV spectrum LED device 100.

One or more actuators 316 may be disposed on the outer surface of the enclosure 102 to control various operational characteristics of the UV spectrum LED device 100. For example, the user may turn on/turn off the UV spectrum LED device 100. Additionally, or alternatively, the user may adjust the lumen output and/or spectrum range of the LEDs 204 to a desired intensity and/or spectrum using an actuator 316. Additionally, or alternatively, the user may control the speed of operation of the one or more fluid moving devices 302 using an actuator 316.

Alternatively, or additionally, a transceiver 318 may be included within the UV spectrum LED device 100. The transceiver 318 is configured to communicate with a remote electronic device using a wireless signal. The wireless transceiver 318 is configured to communicatively couple to a remote electronic device, such as a smart phone, personal computer, other mobile user device, or the like. Any suitable wireless communication signal format may be used, such as, but not limited to, Bluetooth, Wi-Fi, or cellular formats. Alternatively, or additionally, the transceiver may be a wire-based connector that enables a wire-based connection to the remote electronic device. The user may control various operation aspects of the UV spectrum LED device 100 by providing input to an application (AP) or the like residing on their smart phone or mobile device. Control commands received from the user's smart phone or mobile device may be executed by a microcontroller 320.

In some embodiments, an optional pressure equalization membrane, aperture or the like may be disposed in the enclosure 102, the end cap 304, and/or the connector end cap 306.

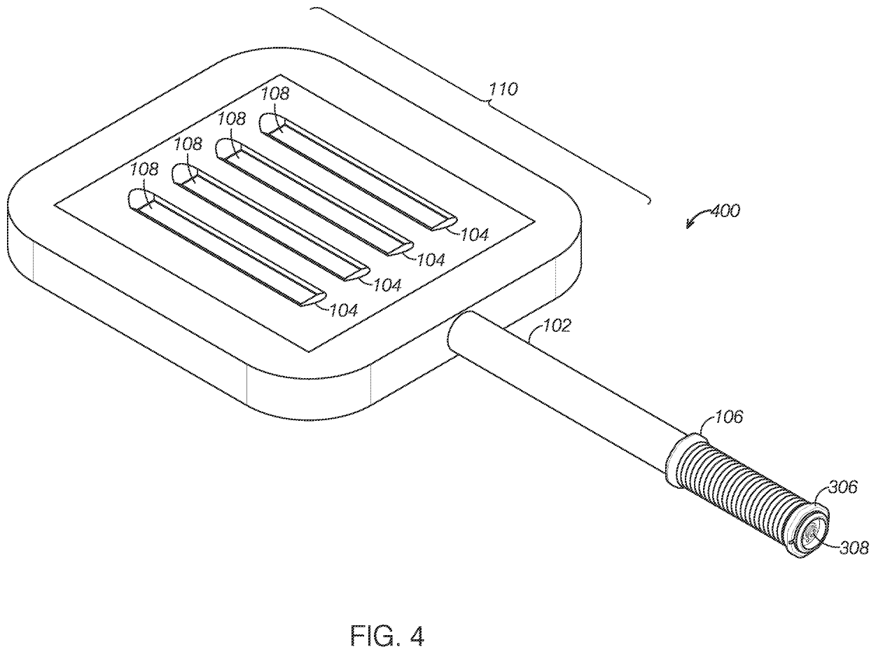

FIG. 4 is a perspective view of a UV spectrum LED device 400 employing four arrays of UV emitting LEDs 204 arranged in parallel with each other proximate to the distal end of the UV spectrum LED device 400. Here, the distal end portion 110 of the enclosure 102 is paddle shaped with four lens housing 104 securing four lens 108 disposed over the arrays of UV emitting LEDs 204 (FIG. 2). This configuration enables the user to disinfect a greater area of a surface with a single use of the UV spectrum LED device 400. The four lens 108 are configured to emit the UV energy generated by the UV emitting LEDs 204 in a single direction. The light conditioning features of the lens 108 may be configured to evenly disperse the generated UV light over the target surface.

Optionally, selected ones of the UV emitting LEDs 204 may be energized to generate a variable intensity of the generated UV light. For example, controlling voltage and/or current applied to the UV emitting LEDs 204 may control UV energy output by the UV emitting LEDs 204.

Any suitable number of UV emitting LEDs 204 and lens 108 may be used in the various embodiments. When a plurality of UV emitting LEDs 204 are secured to the light-emitting module 202, the UV emitting LEDs 204 may be arranged in an array of any width and length of interest using any predefined number of UV emitting LEDs 204. Here, UV energy output from a light-emitting module 202 may be precisely controlled by predefining the number of UV emitting LEDs 204 used in an array, and by predefining the array dimensions. In some embodiments, multiple arrays on a light-emitting module 202 disposed in parallel with each other may be used to control the amount of emitted UV energy. For example, two parallel arrays may reside on a light-emitting module 202. A first predefined amount of UV energy would be emitted when the first array is on (actuated) and the second array is off (deactivated). A second total amount of UV energy would be emitted when both arrays are on (the sum of the first and second predefined amounts of UV energy), if the two arrays are of different sizes or output different amounts of UV energy, a third amount of UV energy would be emitted with the first array is off and the second array is on. Any suitable number of arrays may be used in the various embodiments.

Some UV emitting LEDs 204 may be fabricated as a long filament or a flat strip. Such filament type or strips of UV light emitting diodes are considered as an array of UV emitting LEDs 204 in accordance with this disclosure. Any such variations are intended to be included within the scope of this disclosure and to be protected by the accompanying claims.

Further, each set of UV emitting LEDs 204 and lens 108 may be oriented in different directions and/or be located on other surface areas of the distal end portion 110 of the enclosure 102. Referring to FIG. 4, a plurality of UV emitting LEDs 204 and lens 108 may be disposed on the opposing side of the distal end portion 110 of the enclosure 102. Here, UV light can be concurrently emitted, and/or selectively emitted, in opposing directions. As another example, referring to FIG. 1, a second set of UV emitting LEDs 204 and lens 108 may be disposed on the opposing side of the distal end portion 110 of the enclosure 102. Any such variations are intended to be included within the scope of this disclosure and to be protected by the accompanying claims.

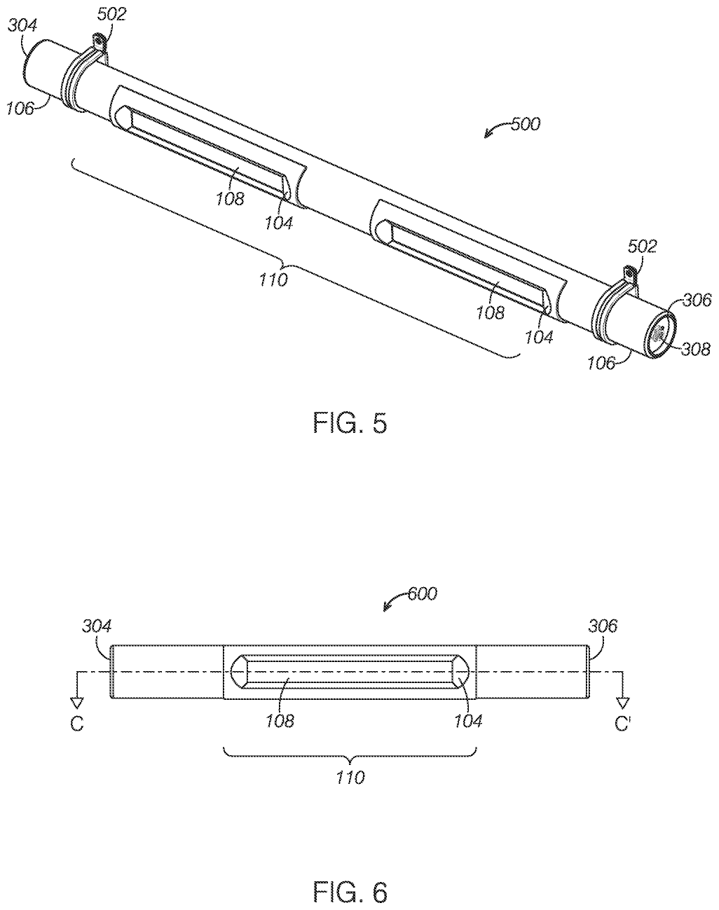

FIG. 5 is a perspective view of a UV spectrum LED device 500 employing two arrays of UV emitting LEDs 204. Here, the first plurality of UV emitting LEDs and the second plurality of UV emitting LEDs are serially aligned along the length of the device proximate to a central portion of the UV spectrum LED device 500. In alternative embodiments, the first plurality of UV emitting LEDs and the second plurality of UV emitting LEDs may be serially aligned along the length of the device proximate at any suitable locations, such as proximate to the distal end of the device

The UV spectrum LED device 500 may be held by hand and is securable to a surface of an object. The UV spectrum LED device 500 includes a grip mechanism 106 for holding device 500 by hand at the distal and proximal ends of the UV spectrum LED device 500. Further, brackets 502 may be used to frictionally secure device 500 to a surface, such as, but not limited to, a wall. In the example shown in FIG. 5, two brackets 502 couple to grip mechanisms 106 of device 500. Alternatively, the UV spectrum LED device 500 may be secured using a single bracket 502. Alternatively, or additionally, a bracket 502 may be used between the two lens housings 104.

Other securing means may be used by alternative embodiments. Holes may be disposed in the UV spectrum LED device 500 for using screws, bolts, wires or the like to secure the UV spectrum LED device 500 to the surface. Clips, straps, snaps, hook and loop fabric, or the like may be used to secure the UV spectrum LED device 500 to a surface. As another non-limiting example, the UV spectrum LED device 500 might be secured to a cleaning device, such as a vacuum, as sweeper, a carpet cleaner, or the like. Any such variations are intended to be included within the scope of this disclosure and to be protected by the accompanying claims.

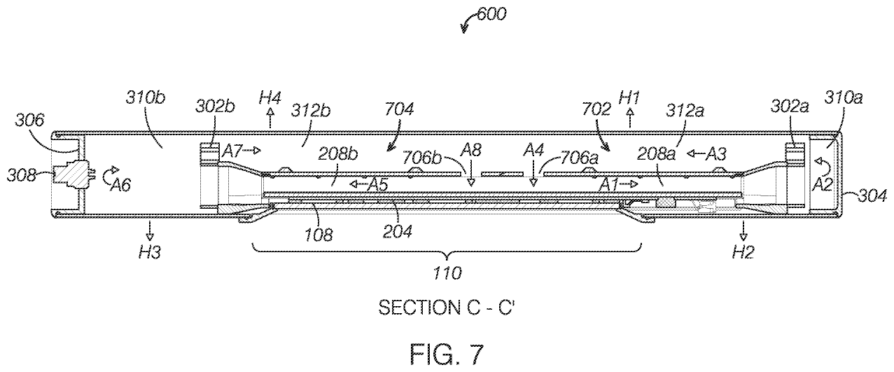

FIG. 6 is a perspective view of a UV spectrum LED device 600 employing one array of UV emitting LEDs and that is securable to a surface of an object. FIG. 7 is a longitudinal cross-sectional view of the UV spectrum LED device 600 of FIG. 6. Elements described hereinabove that are employed in the UV spectrum LED device 600 are not again described for brevity.

The UV spectrum LED device 600 employs two circulating pathways 702 and 704. As the UV emitting LEDs 204 heat the cooling fluid in a first heating passageway 208a (see A1), a first fluid moving device 302a transports the heated fluid into a first transfer passageway 310a (see A2). The heated fluid is then circulated into the first return passageway 312a (see A3). Heat is then transferred out to the ambient environment (see H1). In some embodiments, heat may also be transferred out of the transfer passageway 310a (see H2). The cooled fluid is then returned to the first heating passageway 208a from the return passageway 312a via a port 706a.

Similarly, as the UV emitting LEDs 204 heat the cooling fluid in a second heating passageway 208b (see A5), a second fluid moving device 302b transports the heated fluid into a second transfer passageway 310b (see A6). In some embodiments, heat may be transferred out to the ambient environment (see H3). The fluid is then circulated into the second return passageway 312b (see A7). Heat is then transferred out to the ambient environment (see H4). The cooled fluid is then returned to the second heating passageway 208b from the return passageway 312b via a port 706b.

As shown in FIG. 7 and described above, the cooling fluid flows in opposing circular paths in UV spectrum LED device 600. The A1-A2-A3-A4 circular flow path is counterclockwise and the A5-A6-A7-A8 circular flow path is clockwise. In some examples, the circular flow paths are configured to be in the same direction, such as both clockwise or both counterclockwise.

In the FIG. 5 example, the flow paths are configured differently than shown in FIG. 7. For example, UV spectrum LED device 500 includes ports fluidly connecting the fluid heating passageways with the transfer passageways instead of with the return passageways as depicted in FIG. 7. As a result of the internal configuration of UV spectrum LED device 500, cooling fluid within UV spectrum LED device 500 flows in opposite circular directions than the fluid flow directions of UV spectrum LED device 600 shown in FIG. 7.

In some embodiments, a single port 706 may be used. Alternatively, or additionally, one or more fluid moving devices 302 may be disposed over or within the ports 706a, 706b. Further, differences between the first and second transfer passageways 310 are apparent in FIG. 7 to illustrate various optional configurations of the return passageway 312. Any such variations are intended to be included within the scope of this disclosure and to be protected by the accompanying claims.

It is appreciated in the UV spectrum LED device 600, that only one of the fluid moving device 302a or 302b may be operated to cool the fluid. When more cooling is required, then both of the fluid moving devices 302a, 302b may be concurrently operated.

In the various embodiments, the fluid moving device 302 may be a variable speed fluid moving device 302. Fluid flow output from a variable speed fluid moving device 302 is adjustable by varying the voltage and/or current supplied to the variable speed fluid moving device 302 to vary a circulation speed of the circulating cooling fluid. Any variable speed fluid moving device 302 now known or later developed are intended to be included within the scope of this disclosure and to be protected by the accompanying claims.

In some embodiments, the grip mechanism 106 is configured to be secured to a robot arm of a robotic device. Alternatively, or additionally, the grip mechanism 106 may be configured to be secured to an extension pole or the like. Alternatively, or additionally, the grip mechanism 106 may be configured to be secure to a fixed structure, such as a wall, a ceiling, a floor, a beam, a pole, or the like.

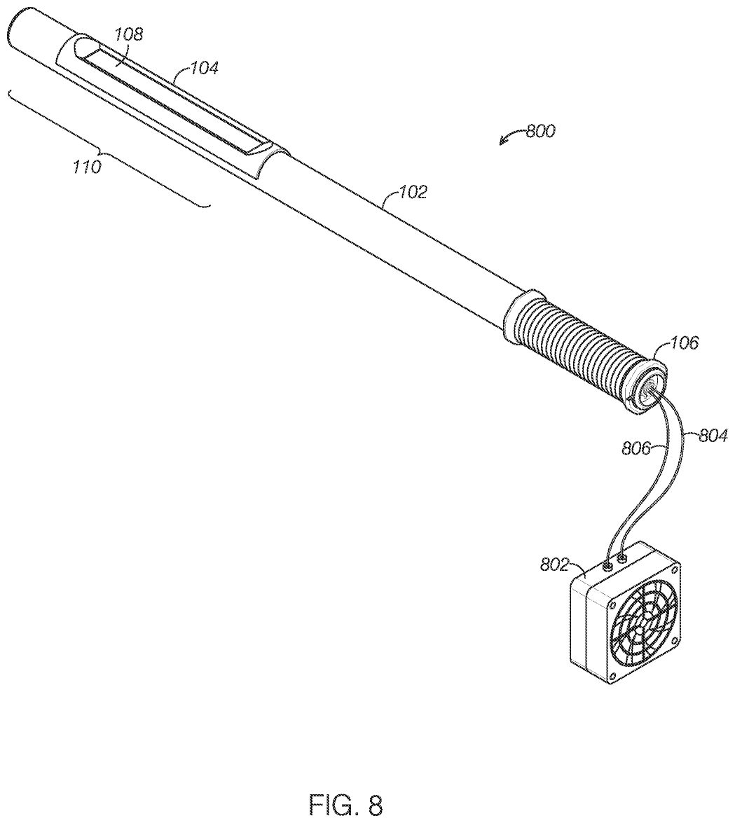

FIG. 8 is a perspective view of a UV spectrum LED device 800 fluidly coupled to a cooling device 802. Elements described hereinabove that are employed in the UV spectrum LED device are not again described for brevity. The end cap 306 is fluidly coupled to the cooling device 802 using a transfer hose connector 804 and a return hose connector 806. The transfer hose connector 804 is in fluid communication with the proximal end of the transfer passageway 310 to transfer the fluid to the cooling device 802. After the fluid is cooled by the cooling device 802, the cooled fluid is returned to the UV spectrum LED device 800 via the return hose connector 806 that is in fluid communication with the proximal end of the return passageway 312.

Any suitable cooling device 802 may be used in the various embodiments of the UV spectrum LED device 800. If air is the cooling medium, then a radiator residing in the cooling device 802 may be used. A fan in the cooling device 802 could move cool ambient air over the radiator. The air cools as it is being circulated through the radiator. Alternatively, the radiator could be surrounded with a cool material, such as cold water, ice, or the like. Some cooling devices 802 may use a refrigeration system. Liquids used for cooling may be similarly cooled.

In an alternative embodiment, the heated air (the coolant fluid after has been heated in the heating passageway 208) may be ejected out from the transfer hose connector 804 into the ambient environment. Cooler fresh ambient air may be taken into the return passageway 312 via the return hose connector 806. In this embodiment, the connectors 304, 306 may be of any suitable length.

In an alternative embodiment, flow of the circulating fluid may be in the opposite direction as described herein above. All such modifications and variations are intended to be included herein within the scope of this disclosure and protected by any later filed claims.

It should be emphasized that the above-described embodiments of the UV spectrum LED device 100 are merely possible examples of implementations of the invention. Many variations and modifications may be made to the above-described embodiments. All such modifications and variations are intended to be included herein within the scope of this disclosure and protected by any later filed claims.

Furthermore, the disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently tiled claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower, or equal in scope to the original claims, are to be considered within the subject matter of the inventions described herein.

1. A UV spectrum LED device, comprising:
an enclosure;
a plurality of UV emitting LEDs residing in the enclosure proximate to a distal end of the UV spectrum LED device;
a lens disposed on a surface of the enclosure,
wherein UV energy emitted by the plurality of UV emitting LEDs passes through the lens onto a surface that is being disinfected;
a heat dissipator configured to receive heat generated by the plurality of UV emitting LEDs; and
a fluid moving device residing in the enclosure,
wherein the enclosure is defined by:
a fluid heating passageway that is proximate to and is in fluid contact with the heat dissipator,
a transfer passageway that is defined by a distal end that is fluidly coupled to a proximal end of the fluid heating passageway, and
a return passageway that is defined by a proximal end that is fluidly coupled to a proximal end of the transfer passageway and that is defined by a distal end that is fluidly coupled to a distal end of the fluid heating passageway,
wherein during operation of the UV spectrum LED device to emit UV energy from the UV spectrum LED device, the fluid moving device operates to circulate a cooling fluid through the fluid heating passageway, the transfer passageway, and the return passageway, and
wherein heat is dissipated from the circulating cooling fluid while in at least one of the transfer passageway and the return passageway.

2. The device of claim 1, wherein the cooling fluid is air.

3. The device of claim 1, wherein the cooling fluid is a liquid.

4. The device of claim 1, wherein the outside of the UV spectrum LED device is hermetically sealed.

5. The device of claim 1, wherein the UV spectrum LED device has a 17:1 ratio for length-to-cross sectional area.

6. The device of claim 1, further comprising:
a first fluid barrier; and
a second fluid barrier,
wherein the first fluid barrier fluidly separates the heating passageway and the return passageway, and
wherein the second fluid barrier fluidly separates the return passageway and the transfer passageway.

7. The device of claim 1, further comprising:
a grip mechanism proximate to a proximal end of the UV spectrum LED device,
wherein the grip mechanism is configured to be grasped by a user.

8. The device of claim 1, further comprising:
a grip mechanism proximate to a proximal end of the UV spectrum LED device,
wherein the grip mechanism is configured to be secured to a surface of an object using a bracket.

9. The device of claim 1, further comprising:
a cooling device configured to cool the cooling fluid; and
an end cap that seals a proximal end of the UV spectrum LED device,
wherein the end cap is fluidly coupled to the cooling device using a transfer hose connector and a return hose connector,
wherein the transfer hose connector is in fluid communication with the proximal end of the transfer passageway to transfer the fluid to the cooling device, and
wherein the cooled fluid is transferred from the cooling device to the return passageway via the return hose connector that is in fluid communication with the proximal end of the return passageway.

10. The device of claim 1, wherein the fluid moving device is a variable speed fluid moving device configured to vary a circulation speed of the circulating cooling fluid.

11. The device of claim 1, wherein:
the fluid moving device is a first fluid moving device;
the fluid heating passageway is a first fluid heating passageway;
the transfer passageway is a first transfer passageway;
the return passageway is a first return passageway;
the enclosure further includes:
a second fluid heating passageway adjacent to the first fluid heating passageway, proximate to the heat dissipater, and in fluid contact with the heat dissipator,
a second transfer passageway that is fluidly coupled to a proximal end of the second fluid heating passageway, and
a second return passageway that is fluidly coupled to the second transfer passageway and that is fluidly coupled to the second fluid heating passageway;
the device further comprises a second fluid moving device residing within the enclosure and separated from the first fluid moving device,
wherein during operation of the UV spectrum LED device to emit UV energy from the UV spectrum LED device, the first fluid moving device operates to circulate a cooling fluid through the first fluid heating passageway, the first transfer passageway, and the first return passageway, and the second fluid moving device operates to circulate a cooling fluid through the second fluid heating passageway, the second transfer passageway, and the second return passageway.

12. The device of claim 11, wherein:
the device further comprises:
a first fluid barrier fluidly separating the first fluid heating passageway from the first return passageway,
a first port defined by the first fluid barrier and fluidly connecting the first return passageway and the first fluid heating passageway,
a second fluid barrier fluidly separating the second fluid heating passageway from the second return passageway, and
a second port defined by the second fluid barrier and fluidly connecting the second return passageway and the second fluid heating passageway;
the first fluid moving device directs fluid flowing through the first return passageway, the first port, and the first fluid heating passageway in a counterclockwise direction; and
the second fluid moving device directs fluid flowing through the second return passageway, the second port, and the second fluid heating passageway in a clockwise direction opposite the counterclockwise direction.

13. The device of claim 12, wherein:
the plurality of UV emitting LEDs is a first plurality of UV emitting LEDs;
the lens is a first lens;
the heat dissipator is a first heat dissipator;
the device further comprises:
a second plurality of UV emitting LEDs spaced from the first plurality of UV emitting LEDs,
a second lens through which UV energy emitted by the second plurality of UV emitting LEDs passes through, and
a second heat dissipator configured to receive heat generated by the second plurality of UV emitting LEDs;
the device is configured to circulate a cooling fluid around the second heat dissipator to dissipate heat generated by the second plurality of UV emitting LEDs in the same manner as the device is configured to circulate a cooling fluid around the first heat dissipator.

14. The device of claim 13,
wherein the surface is a first surface that is being disinfected by the first plurality of UV emitting LEDs,
wherein the second plurality of UV emitting LEDs is on an opposing side of the first plurality of UV emitting LEDs at the distal end of the UV spectrum LED device,
wherein the UV energy emitted by the second plurality of UV emitting LEDs passes through the second lens onto a second surface that is being disinfected, and
wherein the first surface is different from the second surface.

15. The device of claim 13,
wherein the second plurality of UV emitting LEDs is aligned in parallel with the first plurality of UV emitting LEDs at the distal end of the UV spectrum LED device,
wherein the UV energy emitted by the second plurality of UV emitting LEDs passes through the second lens onto the surface that is being disinfected.

16. The device of claim 15,
wherein the UV energy emitted by the first plurality of UV emitting LEDs is a predefined first amount of UV energy,
wherein the UV energy emitted by the second plurality of UV emitting LEDs is a predefined second amount of UV energy,
wherein in response to activation of the first plurality of UV emitting LEDs and deactivation of the second plurality of UV emitting LEDs, the device emits the predefined first amount of UV energy,
wherein in response to deactivation of the first plurality of UV emitting LEDs and activation of the second plurality of UV emitting LEDs, the device emits the predefined second amount of UV energy, and
wherein in response to activation of the first plurality of UV emitting LEDs and activation of the second plurality of UV emitting LEDs, the device emits the predefined first amount of UV energy plus the predefined second amount of UV energy.

17. The device of claim 13,
wherein the first plurality of UV emitting LEDs and the second plurality of UV emitting LEDs are serially aligned along the length of the device proximate to the distal end of the device.

18. The device of claim 13,
wherein the first plurality of UV emitting LEDs and the second plurality of UV emitting LEDs are serially aligned along the length of the device proximate to a central portion of the device.

19. The device of claim 1, wherein the fluid moving device residing in the enclosure is a first fluid moving device, the device further comprising:
a second fluid moving device,
wherein during operation of the UV spectrum LED device to emit UV energy from the UV spectrum LED device, the first fluid moving device and the second fluid moving device can be operated to increase the flow of the circulating cooling fluid.
